(19) United States
(12) Patent Application Publication
(10) Pub. No.: US 2003/0033661 A1
(43) Pub. Date: Feb. 20, 2003

(54) Protective goggle

(76) Inventor: Moon Young Huh, Seoul (KR)

Correspondence Address: KILPATRICK STOCKTON LLP, 607 14TH STREET, N.W., SUITE 900, WASHINGTON, DC, 20005, US

(21) Appl. No.: 10/062,510

(22) Filed: Feb. 5, 2002

(30) Foreign Application Priority Data
Aug. 20, 2001 (KR) KR 2001-25164

(51) Int. Cl. A61F 9/02

(52) U.S. Cl. 2/436

(57) ABSTRACT

Disclosed is an protective goggle for industrial purpose, which permits good ventilation of air, thereby preventing the inner surface of a goggle lens from fogging with moisture of a wearer's perspiration, while efficiently discharging heat at the same time. The protective goggle includes a goggle body, a lens secured at a front portion of the goggle body, and a strap having both ends respectively connected to opposite lateral portions of the goggle body. The protective goggle further includes a pad attached to a rear end of the goggle body, while having a plurality of uniformly spaced protrusions functioning as a cushion. Air inlets each having through holes are formed at lower and lateral portions of the goggle body respectively. Air vent units are mounted on a top surface of the goggle body. Each of air vent units serves to control an amount of air introduced into the interior of the goggle body using a damper. Strap retainers are connected to the goggle body so that they are integral with the goggle body at respective lateral portions of the goggle body. Each of the strap retainers has a groove of a triangular cross section so that it is bent with respect to the goggle body.

### BACKGROUND OF THE INVENTION

1. Field of the Invention

The present invention generally relates to a protective goggle, and more particularly to a protective goggle for industrial purposes, which permits good ventilation of air, thereby preventing the inner surface of a goggle lens from fogging with moisture of a wearer's perspiration, while efficiently discharging heat at the same time.

2. Description of the Related Art

As well known, a protective goggle for industrial purposes is used for protecting eyes of a wearer against toxic spatters, splinters or dusts and the like produced during a procedure such as painting or grinding task.

However, the conventional protective goggle has a fixed size ventilation hole. A large amount of moisture resulting from changes of ambient temperature and the increase of body heat due to working isn't easily discharged through the ventilation hole having a constant size, regardless of the use condition of the goggle. Therefore, the inner surface of the lens of the protective goggle fogs. As a result, there is a problem in that the wearer cannot clearly see a front object.

The goggle fits tightly to the face of the wearer, so that air is trapped between the lens and the face of the wearer. Accordingly, air does not vent, and is kept in this trapped space. It is believed that sealing of the tender parts of human body, particularly, the face for a long time, has a very bad effect on the body, taking into consideration the fact that the wearer's skin respires.

Further, the temperature of the trapped air space rapidly rises above ambient temperature by heat at the wearer's body. When the wearer takes off the goggle, even though the wearer has not moved violently in the state of wearing the goggle for a certain period of time at normal temperatures, hot air is evacuated out of the air space, and the face of the wearer may glow and be flushed.

The conventional protective goggle leaving the characteristics of human body out of consideration has a problem in that it cannot accomplish the purposes of wearing the goggle, such as guaranteed visibility and safety.

### SUMMARY OF THE INVENTION

Therefore, the present invention has been made in view of the above problems, and it is an object of the present invention to provide a protective goggle for industrial purposes, in which air flows smoothly into an interior, while controlling a generation of moisture and efficiently discharging heat at the same time, thereby accomplishing a clear visibility.

It is another object of the present invention to provide a protective goggle for industrial purposes, in which a pad having a cushion of a certain width is attached at the rear end of a goggle body, concretely speaking, at contact end which conforms to the face of a wearer, thereby enhancing fit.

It is a further object of the present invention to provide a protective goggle for industrial purposes, in which strap retainers clamping both ends of a strap are collapsible, thereby readily wearing the strap.

In accordance with the present invention, the above and other objects can be accomplished by providing a protective goggle for industrial purposes comprising a goggle body, a lens secured at a front portion of the goggle body, and a strap having both ends respectively connected to opposite lateral portions of the goggle body, further comprising: a pad attached to a rear end of the goggle body, and having a plurality of uniformly spaced protrusions functioning as a cushion; air inlets formed at lower and lateral portions of the goggle body respectively, each of the air inlets having through holes; air vent units mounted on a top surface of the goggle body, each of the air vent units serving to control an amount of air introduced into the interior of the goggle body using a damper; and strap retainers connected to the goggle body so that they are integral with the goggle body at respective lateral portions of the goggle body, each of the strap retainers having a groove of a triangular cross section so that it is bent with respect to the goggle body.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view illustrating the total construction of a protective goggle for industrial purposes in accordance with the present invention;

FIG. 2 is a cross-sectional view taken along the line A-A of FIG. 1.;

FIG. 3 is a plane view illustrating the protective goggle; and

FIG. 4 is a cross-sectional view illustrating the protective goggle.

### DESCRIPTION OF THE PREFERRED EMBODIMENTS

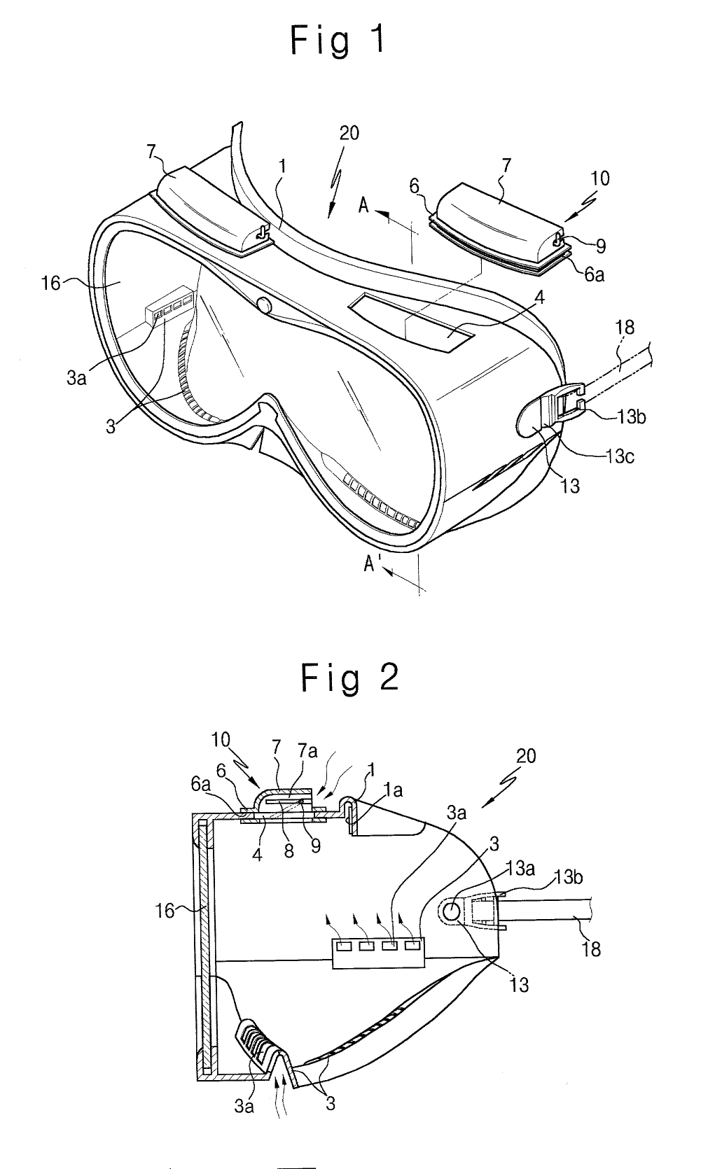

FIGS. 1 to 4 illustrate the total construction of a protective goggle according to the present invention.

Now, the total construction of the protective goggle for industrial purposes according to the present invention will be described with reference to the annexed drawings.

As shown in the drawings, a protective goggle for industrial purposes according to the present invention includes a goggle body 20, a lens 16 secured at a front portion of the goggle body 20, and a strap 18 having both ends respectively connected to opposite lateral portions of the goggle body 20. This configuration of the protective goggle is well known in the technical art. The protective goggle further includes a pad 1 having a certain width and attached to a rear end of the goggle body 20 to enhance fit of the goggle. A plurality of uniformly spaced protrusions 1a are formed at the lower portion of the pad 1, so as to function as a desired cushion upon wearing the goggle.

Air inlets 3 are formed at lower and lateral portions of the goggle body 20, respectively. Each air inlet 3 includes an appropriate number of through holes 3a, thereby allowing air to flow from the outside into the interior of the goggle body 20.

Openings 4 having a desired shape are formed at a top surface of the goggle body 20. Air vent units 10 are fitted in the openings 4, respectively, which are manufactured separately from the goggle body 20. The construction of each air vent unit 10 will be described below.

Each air vent unit 10 includes a frame 6 having, at its outer circumference, a channel 6a adapted to engage with the opening 4 of the goggle body 20, and a hollow cap 7 integral with the frame 6 on the top surface of the frame 6. A hollow space 7a is defined in the hollow cap 7. A shaft 9 extends through the cap 7 and supports a damper 8 adapted to open/close the hollow space 7a, so that the damper 8 can be freely controlled at the outside.

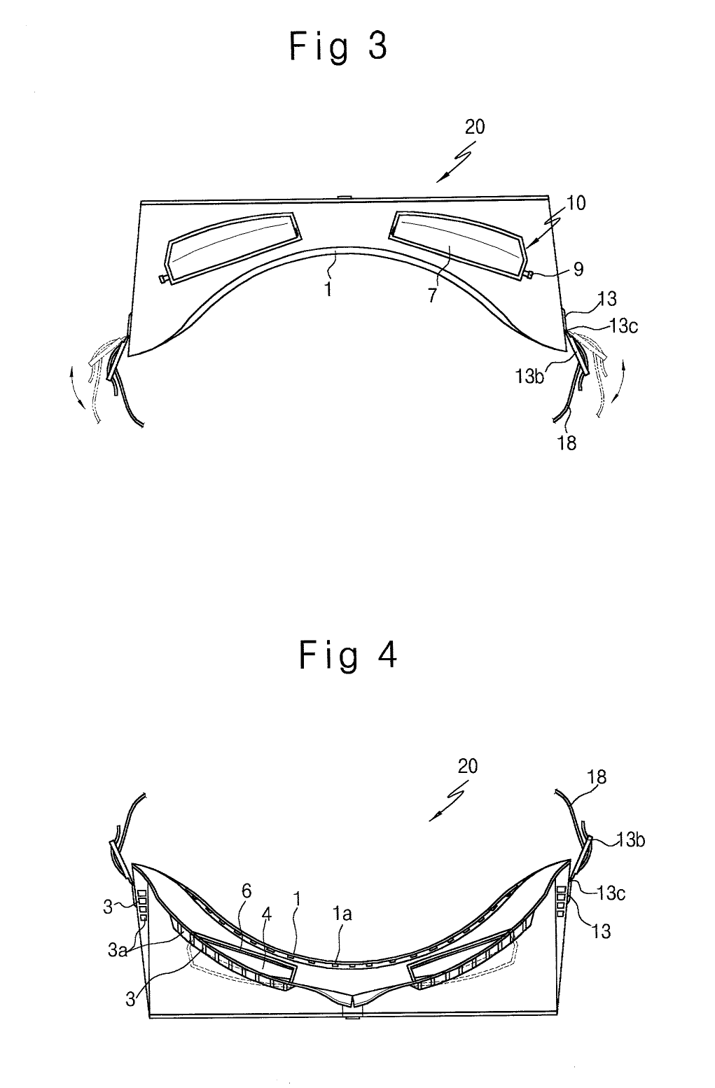

Strap retainers 13 are connected to the goggle body 20 in such a manner that they are integral with the goggle body 20 at opposite lateral portions of the goggle body 20. Each strap retainer has a mounting portion 13a, a retaining portion 13b, and a groove 13c formed between the mounting portion 13a and retaining portion 13b. The mounting portion 13a is attached to the associated lateral portion of the goggle body 20. The retaining portion 13b serves to securely clamp the strap 18. The groove 13c has a triangular cross section, in order to allow the retaining portion 13b to be bent as indicated by the arrows in FIG. 3.

Hereinafter, the operation of the protective goggle having the above-described construction will be described.

When the wearer wears the protective goggle for a painting or grinding task, he can feel a comfortable and secure fit by the cushion function of the pad formed at the rear end of the goggle body. When the lens of the goggle fogs and forward visibility becomes clouded, or when temperature of the trapped air space rises due to body heat, the wearer can control the opening angle of the damper 8 of the air vent unit 10 formed at the top surface of the goggle body 20, to adjust an amount of air introduced and discharged into a interior of the goggle.

Concretely speaking, as the damper 8 closing the hollow space 7a is opened, an amounts of air introduced into the interior of the goggle body can be freely adjusted, thereby adjusting moisture and temperature of the trapped space.

As apparent from the above description, the present invention provides a protective goggle including a pad attached at the rear end of the goggle body of the protective goggle, so as to enhance fit of the goggle. Further, the present invention includes air inlets, each of air inlets has a plurality of through holes and is formed at lower and lateral portions of the goggle body, and air vent units, each of air vent units is mounted on the top surface of the goggle body for freely adjusting an amount of air introduced into a interior of the goggle. This makes it possible to control the generation of moisture of the interior of the goggle and efficiently discharge heat at the same time. The strap retainers respectively attached at the lateral portions of the goggle body can be bent outwardly. Thus, these strap retainers are advantageous in that the wearer can readily wear the protective goggle.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A protective goggle for industrial purposes comprising a goggle body, a lens secured at a front portion of the goggle body, and a strap having both ends respectively connected to opposite lateral portions of the goggle body, further comprising:
a pad attached to a rear end of the goggle body, while having a plurality of uniformly spaced protrusions functioning as a cushion;
air inlets formed at lower and lateral portions of the goggle body respectively, each of the air inlets having through holes;
air vent units mounted on a top surface of the goggle body, each of the air vent units serving to control an amount of air introduced into the interior of the goggle body using a damper; and
strap retainers connected to the goggle body so that they are integral with the goggle body at respective lateral portions of the goggle body, each of the strap retainers having a groove of a triangular cross section so that it is bent with respect to the goggle body.

2. The protective goggle as set forth in claim 1, each of the air vent units comprises:
a frame having a channel at its outer circumference;
a hollow cap integral with the frame on a top surface of the frame, while defining a hollow space defined therein; and
a shaft extending through the hollow space and supporting the damper, thereby controlling an opening angle of the damper.
